(19) United States
(12) Patent Application Publication
(10) Pub. No.: US 2024/0345092 A1
(43) Pub. Date: Oct. 17, 2024

(54) RISK PREDICTION MODEL FOR PROSTATE CANCER

(71) Applicant: RANDOX LABORATORIES LTD., Crumlin Antrim (GB)

(72) Inventors: Stephen Peter Fitzgerald, Crumlin Antrim (GB); Mark Ruddock, Crumlin Antrim (GB); John Lamont, Crumlin Antrim (GB); Declan McKenna, Londonberry (GB)

(21) Appl. No.: 18/682,668

(22) PCT Filed: Aug. 10, 2022
(86) PCT No.: PCT/EP2022/072425

§ 371 Date: Feb. 9, 2024

(30) Foreign Application Priority Data
Aug. 13, 2021 (GB) 2111635.5

(51) Int. Cl. G01N 33/574

(52) U.S. Cl. CPC G01N 33/57434; G01N 33/57488

(57) ABSTRACT

Single markers lack both sensitivity and specificity to stratify risk of PCa in patients who present with elevated tPSA and abnormal DRE. The novel combination of serum markers identified in this study could be employed to help triage patients into ‘low’ and ‘high’ risk categories, allowing general practitioners (GPs) to improve management of patients in primary care settings and potentially reducing the number of referrals for unnecessary, invasive, and costly treatments.

### INTRODUCTION

Prostate cancer (PCa) is very common, with almost 50,000 men diagnosed each year in the UK¹ and 240,000 in the US². Annually, PCa kills almost 35,000 men in the US². Tumours of the prostate are likely to be localised, clinically unapparent, with low Gleason scores³. Slow growing PCa may not cause serious harm⁴. However, prostate cancers that are aggressive have the potential to metastasize and cause serious disease.

Symptoms of PCa include painful or burning sensation during urination, frequent urination (particularly at night (nocturia)), difficulty stopping and starting urination, sudden erectile dysfunction, blood in the urine (haematuria) or semen, bone pain, and weight loss.

Risk factors for PCa include patient age (>50 years), ethnicity (African-American ethnicity and other minority ethnicities have a greater risk of progression and are more likely to develop aggressive cancer than Caucasian men), obesity (patients that are obese have a higher risk of PCa), and family history (blood relative e.g. parent)⁵. Complications of PCa and subsequent treatment include metastatic spread of disease, urinary incontinence and erectile dysfunction⁶.

The gold standard for diagnosing PCa is histological assessment of prostate tissue obtained by transrectal ultrasound-guided systematic (TRUS) core needle biopsy. The most common scale used to evaluate the grade of PCa is the Gleason score⁷. The higher the Gleason score, the more likely that the cancer will grow and spread quickly⁸.

Screening patients for PCa remains controversial and is not recommended, due to the potential for overtreatment⁹. Data presented by the Surveillance, Epidemiological and End Results (SEER) registry have estimated that screening for PCa, using prostate-specific antigen (PSA) alone, resulted in an increase of 28% of patients being over-diagnosed in the US¹⁰. Furthermore, the European Randomised Study of Screening for Prostate Cancer (ERSPC) trial also estimated that when PSA is used alone as a screening tool for PCa, almost 50% of patients were over-diagnosed¹¹.

Although advances in PCa management have been made, an elevated PSA and abnormal digital rectal exam (DRE) would still normally warrant a referral for investigation. As a result, many patients with elevated PSA are referred to secondary care for invasive and costly procedures¹². These are often unnecessary as almost 75% of patients that are referred for further investigation have a negative biopsy¹³. In addition, some 2.5% to 3% of patients are admitted to hospital within a week of their TRUS procedure with serious infection (urinary tract infections and/or bacterial prostatitis). This could be avoided with better decision-making in primary care, but that requires more biological information on the patient's disease to be available to their general practitioner (GP).

Currently, no biomarker or biomarker combinations have been identified that have the sensitivity and specificity to replace PSA¹⁴. Moreover, there is some reluctance to adopt new biomarkers, even though the sensitivity and specificity for PSA is low and the test cannot differentiate between an indolent or aggressive cancer¹⁵. Furthermore, no level of PSA is trulydiagnostic¹⁵. For example, a patient could have a PSA of >10 ng/ml and not have any cancer, whereas another patient with a PSA <1 ng/ml could have aggressive cancer. Therefore, there is an urgent need for new tests which can at least stratify patients and, if possible, be diagnostic. However, it is very unlikely, given the heterogeneous nature of PCa, that a single biomarker will prove to be diagnostic.

Effective management of PCa requires an accurate diagnosis. However, the challenge for the clinician is to differentiate benign conditions (benign prostatic hyperplasia (BPH)) from PCa, which presents with similar symptoms. The PSA test exhibits a negative benefit-to-harm ratio, based on population estimates¹¹. Therefore, biomarkers that would contribute to the sensitivity and specificity of PSA could offer the clinician additional information so that a more informed management decision could be made on whether to refer a patient to secondary care for further investigations or to manage in primary care.

The current invention presents serum biomarker combinations, in patients who present to primary care with PCa-like symptoms, that could be used to improve the triage of patients into low-and high-risk categories, thereby enhancing patient management.

### REFERENCES

1. Cancer Research UK: Prostate Cancer Statistics. https://www.cancerresearchuk.org/health-professional/cancer-statistics/statistics-by-cancer-type/prostate-cancer.
2. Prostate Cancer: Statistics | Cancer.Net. January. https://www.cancer.net/cancer-types/prostate-cancer/statistics. Published 2019.
3. Popiolek M, Rider JR, Andrén O, et al. Natural history of early, localized prostate cancer: A final report from three decades of follow-up. Eur Urol. 2013; 63 (3):428-435. doi:10.1016/j.eururo.2012.10.002.
4. Prostate cancer—Symptoms and causes—Mayo Clinic. https://www.mayoclinic.org/diseases-conditions/prostate-cancer/symptoms-causes/syc-20353087. Accessed Jun. 10, 2021.
5. Hamilton W, Sharp DJ, Peters TJ, Round AP. Clinical features of prostate cancer before diagnosis: A population-based, case-control study. Br J Gen Pract. 2006; 56 (531):756-762.
6. Simoneau AR. Treatment-and disease-related complications of prostate cancer. Rev Urol. 2006; 8 Suppl 2(Suppl 2):S56-67.
7. Rubin MA, Dunn R, Kambham N, Misick CP, O'Toole KM. Should a Gleason score be assigned to a minute focus of carcinoma on prostate biopsy? Am J Surg Pathol. 2000; 24(12):1634-1640. doi:10.1097/00000478-200012000-00007.
8. Epstein JI. Prostate cancer grading: a decade after the 2005 modified system. Mod Pathol. 2018; 31 (S1):47-63. doi:10.1038/modpathol.2017.133.
9. Stark JR, Mucci L, Rothman KJ, Adami HO. Screening for prostate cancer remains controversial. BMJ. 2009; 339. doi:10.1136/bmj.b3601.
10. Etzioni R, Gulati R, Cooperberg M, Penson D, Weiss N, Thompson I. Limitations of basing screening policies on screening trials: The US preventive services task force and prostate cancer screening. Med Care. 2013; 51(4):295-300. doi:10.1097/MLR.0b013e31827da979.
11. Alberts AR, Schoots IG, Roobol MJ. Prostate-specific antigen-based prostate cancer screening: Past and future. 2015. doi:10.1111/iju.12750.
12. Young SM, Bansal P, Vella ET, Finelli A, Levitt C, Loblaw A. Guideline for referral of patients with suspected prostate cancer by family physicians and other primary care providers. Can Fam Physician. 2015; 61(1):33-39.
13. Prostate cancer diagnosis and management Guidance NICE. https://www.nice.org.uk/guidance/ng131/chapter/Recommendations#assessment-and-diagnosis.
14. McNally CJ, Ruddock MW, Moore T, Mckenna DJ. Biomarkers That Differentiate Benign Prostatic Hyperplasia from Prostate Cancer: A Literature Review. 2020. doi:10.2147/CMAR.S250829.
15. Duffy MJ. Biomarkers for prostate cancer: Prostate-specific antigen and beyond. Clin Chem Lab Med. 2020; 58(3):326-339. doi:10.1515/cclm-2019-0693.
16. FitzGerald SP, Lamont J V., McConnell RI, Benchikh EO. Development of a high-throughput automated analyzer using biochip array technology. Clin Chem. 2005; 51(7):1165-1176. doi:10.1373/clinchem.2005.049429.
17. Kurth MJ, McBride WT, Mclean G, et al. Acute kidney injury risk in orthopaedic trauma patients pre and post-surgery using a biomarker algorithm and clinical risk score. Sci Rep. 2020; 10(1):20005-20005. doi:10.1038/s41598-020-76929-y.
18. R Core Team. R: A Language and Environment for Statistical Computing. 2018.

### BRIEF DESCRIPTION OF THE FIGURES

FIG. 1—Prostate cancer model. (A) AUROC for analyte model (AUROC 0.860) and tPSA (AUROC 0.700). When the AUROC for the model (EGF, Log₁₀ IL-8, Log₁₀ MCP-1 and Log₁₀ tPSA) was compared with the AUROC for tPSA, the model significantly improved upon tPSA alone (DeLong p<0.001) at differentiating non-PCa from PCa patients. (B) Simple boxplot of patient score by diagnosis (non-PCa (0) and PCa (1); mean±SD) for the model at a cut off 0.054. (C) Simple scatter with fit line for predicted probability by patient score for the marker model (r=0.95).

### SUMMARY OF THE INVENTION

A first aspect of the current invention is a method of aiding the diagnosis of prostate cancer in a patient presenting with prostate cancer-like symptoms, said method comprising i) determining the level of total prostate-specific antigen (tPSA) and one or more of interleukin 8 (IL-8), monocyte chemoattractant protein 1 (MCP-1), epidermal growth factor (EGF) and neuron-specific enolase (NSE) in an ex vivo blood, serum or plasma sample obtained from the patient and, ii) establishing the significance of the concentration of the biomarkers by inputting each of the biomarker concentration values into a statistical methodology to produce an output that indicates the risk of the patient having or developing prostate cancer. Preferably tPSA levels are determined along with levels of one or both of IL-8 and MCP-1. In one embodiment the levels of tPSA, IL-8, MCP-1 and EGF are measured.

In a further embodiment, the level of one or more of interleukin 10 (IL-10), vascular epidermal growth factor (VEGF), interleukin 1 beta (IL-1β), interleukin 6 (IL-6), soluble tumour necrosis factor receptor 1 (sTNFR1), C-reactive protein (CRP) or D-dimer can also be determined in the patient sample.

The methods of the invention can be enabled by a solid-state device comprising a substrate having an activated surface onto which is immobilised, in discrete areas of said activated surface, one or more binding molecules specific to tPSA and one or more of IL-8, MCP-1 and EGF. One or more binding molecules specific to IL-10, VEGF, IL-1β, NSE, IL-6, sTNFR1, CRP or D-dimer may also be present on the surface of said solid-state device.

An additional aspect of the current invention is the use of serum IL-8 to differentiate between non-prostate cancer conditions and prostate cancer. In one embodiment the non-prostate cancer condition is benign prostatic hyperplasia (BPH).

### DETAILED DESCRIPTION

Almost 70% of men who undergo a prostate biopsy will be negative for PCa. These invasive biopsies subject the patient to potentially serious side effects, i.e., erectile dysfunction and severe sepsis. However, the clinical challenge is to determine when a biopsy is necessary. A risk-based model that can actively triage patients in primary care could significantly reduce the number of patients referred for biopsy. In this study, 19 serum markers potentially involved in PCa were investigated. Results showed 11/16 (68.8%) cytokines were significantly different between the non-PCa vs. PCa group. Seven of these markers were elevated in the PCa group, whereas 4 markers were elevated in the non-PCa group. In the PCa group, 2/3 (66.6%) cancer markers (free PSA and total PSA) were also elevated.

Serum levels of IL-10, EGF, VEGF, MCP-1, sTNFR1, CRP and D-dimer were significantly higher in the PCa patients. Prostate cancer is an inflammatory disease, however, it was found that 4/11 (36.4%) inflammatory markers (IL-8, IL-1β, NSE and IL-6 levels) were significantly lower in the PCa patients. In this study, 19/61 (31.1%) PCa patients (confirmed by histological examination of prostate biopsies) had a tPSA value below the gold standard of 4.0 ng/ml. These PCa patients could have been misdiagnosed within primary care.

The present invention provides a method of aiding the diagnosis of prostate cancer in a patient presenting with prostate cancer-like symptoms, said method comprising determining the concentration of two or more biomarkers selected from the list consisting of tPSA, IL-8, MCP-1, NSE and EGF in an ex vivo sample obtained from the patient; and establishing the significance of the concentration of the biomarkers. Any two, three or four marker combinations of these biomarkers may be useful in aiding the diagnosis of prostate cancer. In a preferred embodiment of the current invention one of the two or more biomarkers is tPSA since this is the gold standard of prostate cancer diagnosis and the current invention provides methods to improve this. Preferred combinations of markers for the diagnosis of prostate cancer include tPSA and IL-8, tPSA and MCP-1, tPSA and EGF, tPSA and NSE, tPSA, EGF and IL-8; tPSA, EGF and MCP-1; tPSA, EGF and NSE; tPSA, IL-8 and MCP-1; tPSA, IL-8 and NSE; tPSA, MCP1 and NSE; tPSA, EGF, IL-8 and MCP1; and tPSA, EGF, IL-8, MCP-1 and NSE. Preferably the combination of markers includes tPSA and one or both of IL-8 and MCP-1, even more preferably the combination of markers consists of tPSA, IL-8, MCP1 and EGF.

A further aspect of the current invention is the measurement of one or more of interleukin 10 (IL-10), vascular epidermal growth factor (VEGF), interleukin 1 beta (IL-1β), interleukin 6 (IL-6), soluble tumour necrosis factor receptor 1 (sTNFR1), C-reactive protein (CRP) or D-dimer in addition to the combinations described previously.

The term “biomarker”, in the context of the current invention, refers to a molecule present in a biological sample of a patient, the levels of which may be indicative of prostate cancer. Such molecules may include peptides/proteins or nucleic acids and derivatives thereof. As used herein the term “determining” means quantitatively analysing for the amount of a substance present, in this case the biomarkers in a patient sample.

The term “tPSA” as used herein refers to total prostate-specific antigen (UniProt P07288) and includes both free and bound PSA.

The term “MCP-1” as used herein refers to monocyte chemoattractant protein 1 (UniProt P13500), also known as chemokine C-C motif ligand 2 (CCL2).

The term “EGF” as used herein refers to epidermal growth factor (UniProt P01133).

The term “IL-8” as used herein refers to interleukin 8 (UniProt P10145).

The term “NSE” as used herein refers to neuron specific enolase (UniProt P09104), also known as gamma enolase and enolase 2 (ENO2).

In the context of the present invention, a “control” or “control value” is understood to mean the level of a biomarker typically found in patients who do not have prostate cancer. The control level of a biomarker may be determined by analysis of a sample isolated from a person who does not have prostate cancer or may be the level of the biomarker understood by the skilled person to be typical for such a person. The control value of a biomarker may be determined by methods known in the art and normal values for a biomarker may be referenced from the literature from the manufacturer of an assay used to determine the biomarker level. The control can be established as a calibration, alternatively, a calibration curve can be generated using analyte preparations at multiple concentrations. The assay signal output generated from a sample can be applied to the calibration curve to enable quantification of the analyte level of said sample.

The “level” of a biomarker refers to the amount, expression level or concentration of the biomarker within the sample. This level can be a relative level in comparison to another biomarker or a previous sample. Biomarker levels may be expressed as ratios, for example ratios between patient levels and control levels for the same biomarker or between levels of different biomarkers within the patient sample.

As used herein, the term “a sample” includes biological samples obtained from a patient or subject, which may comprise blood, plasma, serum, or urine. The methods of the invention described herein are carried out ex vivo. For the avoidance of doubt, the term “ex vivo” has its usual meaning in the art, referring to methods that are carried out in or on a sample obtained from a subject in an artificial environment outside the body of the subject from whom the sample has previously been obtained. The sample may be any sample obtained from the subject from which the biomarkers of the current invention can be determined. Preferred samples include blood samples, serum samples and plasma samples. Most preferably the sample is a serum sample.

The terms “patient” and “subject” are used interchangeably herein and refer to any animal (e.g., mammal), including, but not limited to, humans, non-human primates, canines, felines, rodents, and the like. Preferably, the subject or patient is a male human. More preferably the patient of the current invention is a patient presenting with one or more prostate cancer-like symptoms including painful or burning sensation during urination, frequent urination (particularly at night (nocturia)), difficulty stopping and starting urination, sudden erectile dysfunction, blood in the urine (haematuria) or semen, bone pain, and weight loss. The term “prostate cancer-like symptoms” as used herein is also meant to include a previously determined elevated total PSA result or an abnormal DRE finding.

The concentrations of the biomarkers of the invention may be determined either sequentially or simultaneously in samples previously isolated from patients. The determination of the level of biomarkers in a sample may be determined by routine methods known in the art, such as immunological methods, for example, an immunoturbidimetric assay or ELISA based assay. Preferably, the methods of the present invention use a solid-state device for determining the level of biomarkers in the sample isolated from the patient. The solid-state device comprises a substrate having an antibody that binds specifically to a biomarker immobilised upon it. Such antibodies may be immobilised at discreet areas of an activated surface of the substrate. The solid-state device may perform multi-analyte assays such that the level of one biomarker in a sample isolated from the patient may be determined simultaneously with the level of one or more further biomarkers of interest in the sample. In this embodiment, the solid-state device has a multiplicity of discrete reaction sites each bearing a desired antibody covalently bound to the substrate, and in which the surface of the substrate between the reaction sites is inert with respect to the target biomarker. The solid-state, multi-analyte device may therefore exhibit little or no non-specific binding. Wherein one or more of the biomarkers is not compatible with a multi-analyte format they can be determined simultaneously, or indeed separately, using a suitable format such as ELISA or immunoturbidimetric assay.

A device that may be used in the invention may be prepared by activating the surface of a suitable substrate and applying an array of antibodies on to discrete sites on the surface. If desired, the other active areas may be blocked. The ligands may be bound to the substrate via a linker. In particular, it is preferred that the activated surface is reacted successively with an organosilane, a bifunctional linker and the antibody. A preferred solid support material is in the form of a biochip. A biochip is typically a planar substrate that may be, for example, mineral or polymer based, but is preferably ceramic. The solid-state device used in the methods of the present invention may be manufactured according to the method disclosed in, for example, GB patent number GB2324866. Preferably, the solid-state device used in the methods of the present invention is the Biochip Array Technology system (BAT) (available from Randox Laboratories Limited, Crumlin, Northern Ireland). More preferably, the Evidence Evolution, Evidence Investigator and Multistat apparatus (also available from Randox Laboratories) may be used to determine the levels of biomarkers in the sample.

The solid-state device comprises binding molecules attached thereto, said binding molecules having affinity specific for tPSA and, separately, one or more of IL-8, MCP-1, EGF, and NSE. Preferably the binding molecules have affinity for tPSA and, separately, one or both of IL-8 and MCP-1. Even more preferably the binding molecules, each in discrete locations, have affinity specific for tPSA, IL-8, MCP1 and EGF.

The solid-state device may further comprise, each in discrete locations, one or more binding molecules each having affinity specific for an additional biomarker selected from one or more of interleukin 10 (IL-10), vascular epidermal growth factor (VEGF), interleukin 1 beta (IL-1β), interleukin 6 (IL-6), soluble tumour necrosis factor receptor 1 (sTNFR1), C-reactive protein (CRP) or D-dimer.

The present invention also provides the use of the solid-state device described in a method for aiding in the diagnosis of prostate cancer in a patient.

The terms “immunoassay”, “immuno-detection” and immunological methods” are used interchangeably herein and refer to antibody-based techniques for identifying the presence of or levels of a protein in a sample. Examples of such assays and methods are well known to those of skill in the art.

The term “binding molecule” as used herein refers to refers to any molecule that is capable of specifically binding to a target molecule, in this case the biomarkers, such that the target molecule can be detected as a consequence of said specific binding. Binding molecules that can be used in the present invention include, for example, antibodies, aptamers, phages and oligonucleotides. In a preferred embodiment of the current invention the binding molecules are antibodies. The term “antibody”, or the plural thereof, refers to an immunoglobulin which specifically recognises an epitope on a target as determined by the binding characteristics of the immunoglobulin variable domains of the heavy and light chains (VHS and VLS), more specifically the complementarity-determining regions (CDRs). Many potential antibody forms are known in the art, which may include, but are not limited to, a plurality of intact monoclonal antibodies or polyclonal mixtures comprising intact monoclonal antibodies, antibody fragments (for example Fab, Fab′, and Fr fragments, linear antibodies, single chain antibodies and multi-specific antibodies comprising antibody fragments), single chain variable fragments (scFv's), multi-specific antibodies, chimeric antibodies, humanised antibodies and fusion proteins comprising the domains necessary for the recognition of a given epitope on a target. Preferably, references to antibodies in the context of the present invention refer to polyclonal or monoclonal antibodies. Antibodies may also be conjugated to various reporter moieties for a diagnostic effect, including but not limited to radionuclides, fluorophores, or dyes.

The term “binds specifically”, in the context of antibody-epitope interactions, refers to an interaction wherein the antibody and epitope associate more frequently or rapidly, or with greater duration or affinity, or with any combination of the above, than when either antibody or epitope is substituted for an alternative substance, for example an unrelated protein. Generally, but not necessarily, reference to binding means specific recognition. Techniques known in the art for determining the specific binding of a target by a monoclonal antibody or lack thereof include but are not limited to, FACS analysis, immunocytochemical staining, immunohistochemistry, western blotting/dot blotting, ELISA, affinity chromatography. By way of example and not limitation, specific binding, or lack thereof, may be determined by comparative analysis with a control comprising the use of an antibody which is known in the art to specifically recognise said target and/or a control comprising the absence of, or minimal, specific recognition of said target (for example wherein the control comprises the use of a non-specific antibody). Said comparative analysis may be either qualitative or quantitative. It is understood, however, that an antibody or binding moiety which demonstrates specific recognition of a given target is said to have higher specificity for said target when compared with an antibody which, for example, specifically recognises both the target and a homologous protein.

A biomarker present in a sample isolated from a patient having cancer may have levels which are different to that of a control. However, the levels of some biomarkers that are different compared to a control may not show a strong enough correlation with cancer such that they may be used to diagnose cancer with an acceptable accuracy. If two or more biomarkers are to be used in the diagnostic method a suitable mathematical or machine learning classification model, such as logistic regression equation, can be derived. Such models as described herein may be referred to as “statistical methodologies”. The significance of the levels of the biomarkers can be established by inputting into said model. Such a classification model may be chosen from at least one of decision trees, artificial neural networks, logistic regression, random forests, support vector machine or indeed any other method developing classification models known in the art. The output of the models used herein would correlate with the risk of a patient having or developing prostate cancer. Such an output could be a numerical value, for example a number between 0 and 1, an odds ratio value, a risk ratio/relative risk value or an alphabetic output such as ‘yes’ or ‘no’ or ‘high risk’, ‘low risk’ etc.

Variables can be logarithmically transformed in a regression model when data is not normally distributed. In one embodiment of the current invention the values for IL-8, MCP-1 and tPSA were log₁₀ transformed in a combination of EGF, log₁₀ IL-8, log₁₀ MCP-₁, and log₁₀ tPSA which significantly improved the predictive potential of tPSA alone to identify patients with PCa. This marker combination had an increased AUROC (0.860 vs. 0.700), sensitivity (78.7% vs. 68.9%), specificity (76.5% vs. 67.2%), positive predictive value (PPV) (76.2% vs. 66.7%) and negative predictive value (NPV) (79.0% vs. 69.4%) compared to tPSA. The skilled person will appreciate that the model generated for a given population may need to be adjusted for application to datasets obtained from different populations or patient cohorts.

The term “sensitivity”, used in the context of a diagnostic test, describes the percentage or ratio of subjects actually positive for the condition that are deemed positive by the biomarker test, sometimes referred to as the true positive rate. The term “specificity”, used in the context of a diagnostic test, indicates the percentage or ratio of the subjects deemed negative by the biomarker test that are actually negative for the condition (true negative rate). In these studies, it is customary for the number of positive subjects to be pre-determined by the current gold standard of testing (in this case, histopathology of biopsied tumour tissue), in order that these analyses may be performed.

One convenient goal to quantify the diagnostic accuracy of a laboratory test is to express its performance by a single number. The most common global measure is the area under the curve (AUC) of the receiver-operating characteristics (ROC) plot. The area under the ROC curve is a measure of the probability that the perceived measurement will allow correct identification of a condition. By convention, this area is typically ≥0.5. Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0). In the context of the present invention, the two different conditions are whether a patient has prostate cancer or not. Table 4 shows the areas under the curve for tPSA on its own and in combinations with the other markers. In a clinical setting it would be desirable to assign prostate cancer with 100% sensitivity. This means that the majority of subjects, who will end up outside of this category, can have prostate cancer ruled out and avoid an unnecessary biopsy.

Using a combination of the identified markers (EGF, IL-8 and MCP-1) with tPSA, an algorithm to triage patients suspected of PCa who present in primary care was developed. The PCa model was developed using statistical analyses and mathematical modelling of data generated from biochip assays. Using LASSO modelling, serum markers were identified that contributed to accuracy whilst simultaneously reducing the over-fitting of the model. The PCa model underwent rigorous testing using resampling methods to determine its viability in this patient cohort. A cut-off value was selected for the patient risk score output which maximised sensitivity and specificity for this dataset. It is well understood in the art that biomarker normal or ‘background’ concentrations may exhibit slight variation due to, for example, age, gender, or ethnic/geographical genotypes. As a result, the cut-off value used in the methods of the invention may also vary due to optimization depending upon the target patient or population. Adjusting the cut-off will also allow the operator to increase the sensitivity at the expense of specificity and vice versa. In addition, the model was tested using a Dynamic Nomogram that can simply, and effectively, visualize a patient's risk of PCa. The data for each marker is entered into nomogram and the App returns a numerical output; 0 to 1 probability (i.e., non-PCa and risk of PCa).

The PCa model could stratify presenting patients into low and high-risk categories based on a biomarker risk score (BRS). The PCa model outperformed tPSA alone at stratifying non-PCa patients from those with PCa. It is proposed that the PCa model could be used clinically to allow clinicians to make evidence-based decisions regarding patient management, i.e., when to refer a patient to secondary care for biopsy.

Multivariate approaches and modelling to developing a risk prediction model offer an advantage in accuracy compared to that of a single marker. Combining proteomic, genomic, and clinical measurements provide evidence-based decision making for the clinician. These risk stratification methods are also recommended by the National Institute for Health and Care Excellence (NICE 2019) guidelines for PCa.

Furthermore, utilizing the methods of the current invention there is the potential to input clinical characteristics in addition to biomarker results which could be combined into a single ‘risk score’ or be presented as a BRS and separate clinical risk score (CRS). Currently, several clinical risk factors for PCa, e.g., age, prostate volume, and family history of PCa, have been identified. Incorporating clinical risk with results from the biomarkers would allow clinicians to make evidence-based decisions and assist with patient management. Our research suggests that the PCa models could be improved further with the collection of clinical factors such as age, family history of PCa, BMI levels, and other clinical factors. For example, patients who are positive for both a biomarker risk score (as determined by any of the biomarker combinations presented herein) and a clinical risk score would be placed into a higher risk category than patients who are only positive for one of the two risk scores. Therefore, a further embodiment of the current invention is the combination of a BRS and a CRS to categorise patients presenting with prostate cancer-like symptoms into risk groups. The models are not designed to replace tPSA but to acknowledge its limitations and potentially provide clinicians with additional management evidence when recommending which patients should be referred for biopsy.

Circulating IL-8 serum levels have not previously been shown to be a significant predictor of diagnosis, aggressiveness, or prognosis for PCa. However, increased circulating IL-8 serum levels have been detected in patients with underlying inflammatory disease. In our study, IL-8 was identified as a marker that could differentiate non-PCa from PCa, potentially by identifying patients with inflammatory disease i.e., BPH. Surprisingly, IL-8 levels were significantly lower in prostate cancer patients compared to non-prostate cancer controls. Almost 50% of non-prostate controls had BPH (30/64), IL-8 levels in prostate cancer patients were also significantly lower when compared only to BPH patients (BPE 143.60+257.38 pg/ml (n=30) vs. PCa (n=61) 28.35+42.39 pg/ml), with an AUROC of 0.671 (Confidence intervals—0.555-0.787).

As such, a further aspect of the current invention is the use of serum IL-8 to aid in the differentiation between non-prostate cancer conditions and prostate cancer. Preferably the non-prostate condition is BPH.

### Materials and Methods

### Patient Cohort and Sample Collection

One hundred and twenty-five patients were included in the study. The patient cohort consisted of two independent patient sample sets.

The first set of patients (N=33; n=10 non-PCa and n=23 PCa) were recruited by Royal Surrey County Hospital, Frimley Park Hospital, Wexham Park Hospital and Basingstoke and North Hampshire Hospital urology clinics between 2015 and 2018 (ProCure Study 170858, Diagnosis of Clinically Significant Prostate Cancer; ethics reference: 15/LO/0218). Inclusion criteria included men >18 years referred by their GP to investigate the cause of an abnormal PSA test. Exclusion criteria included an active urine infection, confirmed by urine dipstick testing or midstream urine microscopy, men with a PSA <4 and >20 ng/ml, men already diagnosed with PCa, men with a prior or concurrent malignancy (apart from basal cell carcinoma of the skin), and men who cannot give informed consent. Blood (24 ml) and urine (20-30 ml) were collected after prostatic examination, along with a detailed clinical history. The study complied with the Declaration of Helsinki and written informed consent was obtained from all participants.

The second patient cohort (N=92; n=54 non-PCa and n=38 PCa) was obtained from Discovery Life

Sciences (DLS), California, USA. Patient samples were de-identified and publicly available and were thus exempt from the requirement of the Institutional Review Board (IRB) approval (Exempt Category 4, IRB/EC). However, samples were procured pursuant to informed consent provided by the individual under approved protocols 45 CFR 46.116. Serum (1 ml) with clinical history was obtained for each DLS patient. Samples were selected from treatment naive patients based on ICD-10 codes for prostate-related conditions.

### Pathological Examination of Prostate Biopsies

Prostate cancer was confirmed by histological examination of prostate biopsies from both sample sets. Gleason scores assigned by pathologists are described in Table 1. The non-PCa group included patients with confirmed benign prostatic hyperplasia (BPH) (n=30/61 (49.2%)). All patients were treatment naive at the time of prostate biopsy.

Both patient cohorts were combined (N=125) and separated into two groups, depending on pathology reports: non-PCa (n=64/125 (51.2%)) and PCa (n=61/125 (48.8%)).

### Clinical Factors and Behaviours

Clinical factors were not available for all patients. However, where data was available, the most common presenting symptoms included: BPH, lower urinary tract symptoms (LUTS), urine retention, urgency of urination, nocturia, lower back pain, microscopic haematuria, hyperlipidaemia, and hypertension. For many of the patients, there was no previous history of benign disease prior to their PCa diagnosis.

Smoking history and alcohol consumption (units/week) were also available for a limited number of patients. Many PCa patients were former smokers. Where data was available, the number of cigarettes smoked per day ranged from 10-25. Pack year data was not available. Alcohol consumption ranged from 1 to 48 units/week (where data was available).

Medications were also noted for a limited number of patients; where data was available, the most common drugs that the patents were prescribed included: Sertraline, Loratadine, Omeprazole, Aspirin, Tamsulosin, Simvastatin, Losartan, Atorvastatin, Imvastatin, Bendroflumethiazide, Citalopram, Sildenafil, Fluoxetine, Ranitidine, Metformin and Bisoprolol.

### Biomarker Analysis

Patient samples were analysed in duplicate by Randox Laboratory Clinical Services (RCLS), Antrim, UK by scientists blinded to patient data. In total, 19 biomarkers were investigated by Biochip Array Technology (BAT) (Randox Laboratories Ltd, Crumlin, UK)¹⁶ using the Evidence Investigator analyser (Randox Laboratories Ltd, Crumlin, UK), following manufacturer's instructions. The limits of detection (LOD) for the markers on the biochip arrays were: EGF 2.5 pg/ml, IFNγ 2.1 pg/ml, IL-1α 0.9 pg/ml, IL-1β 1.3 pg/ml, IL-2 4.9 pg/ml, IL-4 3.5 pg/ml, IL-6 0.4 pg/ml, IL-8 2.3 pg/ml, IL-10 1.1 pg/ml, MCP-1 25.5 pg/ml, TNFα 3.7 pg/ml, VEGF 10.8 pg/ml, CRP 0.67 mg/l, D-dimer 2.1 ng/ml, NSE 0.26 ng/ml and sTNFR1 0.24 ng/ml. CEA 0.29 ng/ml, fPSA 0.02 ng/ml and tPSA 0.045 ng/ml. Biomarkers below the LOD were recorded as 90% of the LOD17.

Statistical Analyses

Statistical analyses were undertaken using R version 4.0.5¹⁸. Wilcoxon rank sum test was used to identify differentially expressed markers. Markers with a p<0.05 were considered significant. The ability of the markers to predict PCa was further investigated using logistic Lasso regression, following cross-validation testing of several models. For individual markers and marker combinations, areas under the receiver operator characteristic (AUROC) (and 95% Cl), sensitivity (and 95% Cl), specificity (and 95% Cl), positive predictive value (PPV) and negative predictive value (NPV) were calculated to identify models that differentiated between the two diagnostic groups (non PCa vs. PCa). The DeLong test was used to compare AUROCs for the model and tPSA; a p<0.05 was considered significant.

### Results

Clinical and pathological characteristics of the patients involved in the study are described in Table 1. Both tPSA and fPSA were significantly elevated in the PCa group. However, CEA was not significantly different.

TABLE 1
Clinical and pathological characteristics of the patients. Data
shown as mean ± standard deviation (SD) or n/total (%),
Wilcoxon rank sum test; p < 0.05 was considered significant.

| Clinical | Non-PCa | PCa | |
|---|---|---|---|
| Characteristics | (n = 64) | (n = 61) | p value |
| Age (years) | 62.7 ± 10.4 | 64.4 ± 8.3 | 0.439 |
| BPH | 30/64 (46.9%) | | |
| Gleason Score | | | |
| 6 | | 11/60 (18.3%) | |
| 7 | | 31/60 (51.7%) | |
| 8 | | 12/60 (20%) | |
| 9 | | 6/60 (10%) | |
| tPSA (ng/ml) | 4.2 ± 3.7 | 20.8 ± 58.2 | <0.001 |
| fPSA (ng/ml) | 0.8 ± 0.9 | 3.6 ± 9.5 | 0.005 |
| CEA (ng/ml) | 2.4 ± 3.0 | 4.4 ± 16.5 | 0.158 |

### Biochip Array Technology

From the marker results obtained using the biochip arrays, 11/16 (68.8%) markers were significantly different between the non-PCa and PCa patient groups (Table 2). Of these 7/16 (43.8%) markers, including MCP-1 and EGF, were significantly elevated in the PCa patients vs. non-PCa; 4/16 (25%), including IL-8, were significantly lower in the PCa vs. non-PCa, and 5/16 (31.2%) were not significantly different between either group.

TABLE 2
Analysis showed 11/16 (68.8%) serum markers were significantly
different between the non-PCa and PCa patient group.
Data shown as mean ± SD. Wilcoxon rank sum test;
p < 0.05 was considered significant.

| | non-PCa | PCa | |
|---|---|---|---|
| Marker | (n = 64) | (n = 61) | p value |
| IL-8 (pg/ml) | 175.3 ± 261.5 | 28.4 ± 42.4 | <0.001 |
| IL-10 (pg/ml) | 1.8 ± 2.0 | 3.2 ± 9.0 | <0.001 |
| MCP-1 (pg/ml) | 189.9 ± 106.9 | 291.1 ± 148.0 | <0.001 |
| VEGF (pg/ml) | 69.1 ± 68.5 | 145.5 ± 132.9 | <0.001 |
| IL-1β (pg/ml) | 11.6 ± 44.1 | 1.9 ± 1.2 | 0.001 |
| NSE (ng/ml) | 15.3 ± 11.3 | 7.8 ± 5.3 | 0.001 |
| EGF (pg/ml) | 87.1 ± 54.7 | 129.5 ± 81.8 | 0.002 |
| IL-6 (pg/ml) | 37.8 ± 148.2 | 19.9 ± 42.1 | 0.004 |
| sTNFRI (ng/ml) | 1.2 ± 1.3 | 1.5 ± 1.1 | 0.009 |
| CRP (μg/ml) | 45.5 ± 41.0 | 73.8 ± 49.6 | 0.012 |
| D-dimer (ng/ml) | 173.6 ± 194.2 | 331.0 ± 382.9 | 0.014 |
| IL-1α (pg/ml) | 0.8 ± 0.1 | 0.8 ± 0.0 | 0.090 |
| TNFα (pg/ml) | 4.2 ± 3.1 | 3.9 ± 1.4 | 0.130 |
| IL-2 (pg/ml) | 4.7 ± 1.6 | 4.4 ± 0.1 | 0.327 |
| IFNγ (pg/ml) | 1.9 ± 0.2 | 1.9 ± 0.2 | 0.606 |
| IL-4 (pg/ml) | 3.2 ± 0.4 | 3.2 ± 0.4 | 0.608 |

### Regression Analysis

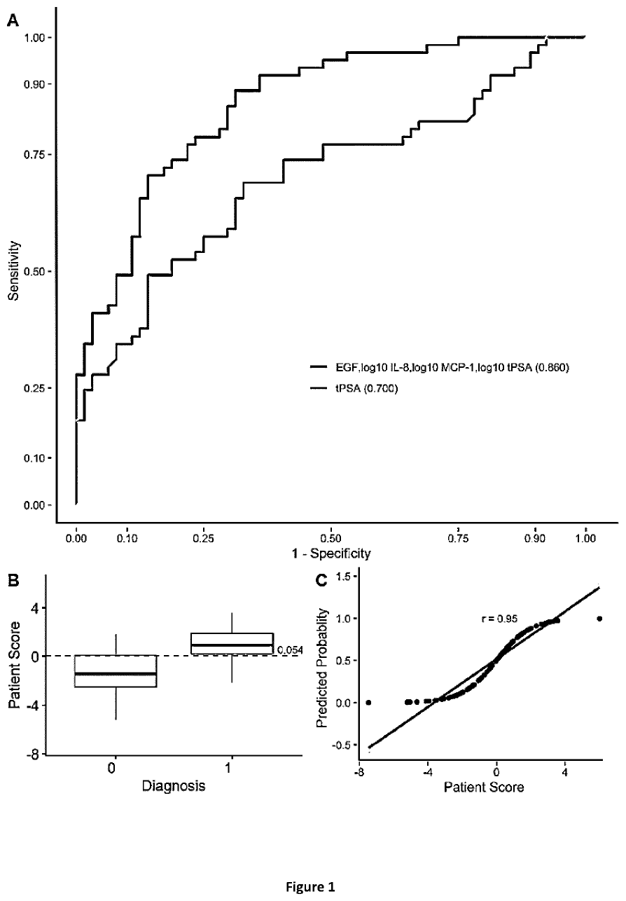

Logistic Lasso regression identified a model for a combination of markers that demonstrated higher sensitivity and specificity vs. tPSA alone (Table 3). The four markers selected by Lasso regression to identify patients with PCa included EGF, IL-8, MCP-1 and tPSA (FIG. 1A). As some of the data was not normally distributed, log₁₀ transformation was applied to IL-8, MCP-1 and tPSA in the model.

When comparing the new model identified by Lasso (EGF+log₁₀ IL-8+log₁₀ MCP-1+log₁₀ tPSA) to tPSA on its own, the number of false positives were reduced from 21/64 (32.8%) to 15/64 (23.4%).

TABLE 3
Individual analytes and model EGF, IL-8, MCP-1 and tPSA AUROC,
sensitivity, specificity, PPV and NPV for non-PCa vs. PCa.

| Markers and Marker | AUROC | Sensitivity | Specificity | PPV | NPV |
|---|---|---|---|---|---|
| Combination | (95% CI) | (95% CI) | (95% CI) | (%) | (%) |
| EGF | 0.658 | 0.656 | 0.609 | 61.5 | 65.0 |
| | (0.562-0.754) | (0.541-0.770) | (0.500-0.734) | | |
| IL-8 | 0.703 | 0.738 | 0.563 | 61.6 | 69.2 |
| | (0.612-0.794) | (0.623-0.836) | (0.438-0.688) | | |
| MCP-1 | 0.739 | 0.738 | 0.703 | 70.3 | 73.8 |
| | (0.651-0.826) | (0.623-0.836) | (0.594-0.813) | | |
| tPSA | 0.700 | 0.689 | 0.672 | 66.7 | 69.4 |
| | (0.606-0.793) | (0.574-0.803) | (0.563-0.781) | | |
| EGF + log₁₀ IL-8 + | 0.860 | 0.787 | 0.765 | 76.2 | 79.0 |
| log₁₀ MCP-1 + log₁₀ tPSA | (0.796-0.923) | (0.688-0.885) | (0.656-0.875) | | |

### Calculating Patient Risk Score (PRS)

Risk of PCa was based on the following marker combination: EGF, log₁₀ IL-8, log₁₀ MCP-1 and log₁₀ tPSA. An equation of PRS=−8.961+(0.010*EGF)+(−1.524*log₁₀ IL-8)+(3.958*log₁₀ MCP-1)+(1.315*log₁₀ tPSA) was derived for this dataset, a cut-off 0.054 (as shown in FIG. 1B) was applied to achieve the highest sensitivity and specificity for identifying patients with PCa; PRS<0.054, patients are negative for PCa, whereas PRS≥0.054, patients would be positive for PCa. It should be noted that the PRS (also referred to as biomarker risk score) would be used in combination with clinical risk factors when triaging patients. Thus, patients with a positive risk score and positive clinical risk factors (e.g., painful or burning sensation during urination, frequent urination, difficulty starting or stopping urination, sudden erectile dysfunction, blood in urine or semen) would be prioritised for urgent referral for further investigations. Patients that were positive for clinical risk factors and negative for BRS could potentially be managed in primary care, or referred for investigation, as necessary. Importantly, this type of combined measurement approach is recommended for risk stratification methods by the National Institute for Health and Care Excellence (NICE 2019) guidelines for PCa.

To test the linearity of the model, predicted probability was plotted against patient score (FIG. 1C). The high correlation between the predicted probability and patient score (r=0.95) would suggest confidence in the model.

TABLE 4
Area under the curve values for tPSA and biomarker combinations.

| | AUC | | | | |
|---|---|---|---|---|---|
| Biomarker(s) | value | +EGF | +IL8 | +MCP1 | +NSE |
| tPSA | 0.700 | 0.769 | 0.796 | 0.804 | 0.779 |
| tPSA + EGF | 0.769 | — | 0.847 | 0.838 | 0.842 |
| tPSA + IL8 | 0.796 | 0.847 | — | 0.859 | 0.806 |
| tPSA + MCP1 | 0.804 | 0.838 | 0.859 | — | 0.828 |
| tPSA + NSE | 0.779 | 0.842 | 0.806 | 0.828 | — |
| tPSA + EGF + IL8 | 0.847 | — | — | 0.881 | 0.864 |
| tPSA + EGF + MCP1 | 0.838 | — | 0.881 | — | 0.869 |
| tPSA + EGF + NSE | 0.842 | — | 0.864 | 0.869 | — |
| tPSA + IL8 + MCP1 | 0.859 | 0.881 | — | — | 0.858 |
| tPSA + IL8 + NSE | 0.806 | 0.864 | — | 0.858 | — |
| tPSA + MCP1 + NSE | 0.828 | 0.869 | 0.858 | — | — |
| tPSA + EGF + IL8 + MCP1 | 0.881 | — | — | — | 0.890 |

1. A method of aiding the diagnosis of prostate cancer in a patient presenting with prostate cancer-like symptoms, said method comprising i) determining the level of total prostate-specific antigen (tPSA) and one or more of interleukin 8 (IL-8), monocyte chemoattractant protein 1 (MCP-1), epidermal growth factor (EGF) and neuron-specific enolase (NSE) in an ex vivo blood, serum or plasma sample obtained from the patient and, ii) establishing the significance of the concentration of the biomarkers by inputting each of the biomarker concentration values into a statistical methodology to produce an output that indicates the risk of the patient having or developing prostate cancer.

2. The method of claim 1, wherein the levels of any of the following combinations are determined:
i) tPSA and IL-8;
ii) tPSA and MCP-1;
iii) tPSA and EGF;
iv) tPSA and NSE;
v) tPSA, IL-8 and MCP-1;
vi) tPSA, IL-8 and EGF;
vii) tPSA, MCP-1 and EGF;
viii) tPSA, EGF and NSE;
ix) tPSA, MCP-1 and NSE;
x) tPSA, IL-8 and NSE;
xi) tPSA, MCP-1, IL-8 and EGF; or xii) tPSA, MCP-1, IL-8, EGF and NSE.

3. The method of claim 1, wherein the level of one or more of interleukin 10 (IL-10), vascular epidermal growth factor (VEGF), interleukin 1 beta (IL-1β), interleukin 6 (IL-6), soluble tumour necrosis factor receptor 1 (STNFR1), C-reactive protein (CRP) or D-dimer is also determined in the patient sample.

4. The method of claim 2 wherein the levels of tPSA, MCP-1, IL-8 and EGF are determined.

5. A solid-state device comprising a support material and binding molecules attached thereto, said binding molecules having affinity specific for tPSA and, separately at least one of IL-8 and MCP-1, with the binding molecules for each being in discrete locations on the support material.

6. The solid-state device of claim 5 wherein the binding molecules, separately, have affinity for tPSA, IL-8, MCP-1 and EGF.

7. A method to differentiate between non-prostate cancer conditions and prostate cancer, the method comprising measuring the amount of serum IL-8 in a subject.

8. The method of claim 7, wherein the non-prostate cancer condition is benign prostatic hyperplasia (BPH).

9. The method of claim 2, wherein the level of one or more of interleukin 10 (IL-10), vascular epidermal growth factor (VEGF), interleukin 1 beta (IL-1β), interleukin 6 (IL-6), soluble tumour necrosis factor receptor 1 (sTNFR1), C-reactive protein (CRP) or D-dimer is also determined in the patient sample.

10. The method of claim 9, wherein the levels of tPSA, MCP-1, IL-8 and EGF are determined.
